(19) United States
(12) Patent Application Publication
(10) Pub. No.: US 2021/0241773 A1
(43) Pub. Date: Aug. 5, 2021

(54) VOICE RECORDING & LABEL MAKING SYSTEM

(71) Applicant: Frank Appiah, New Albany, OH (US)

(72) Inventor: Frank Appiah, New Albany, OH (US)

(21) Appl. No.: 16/587,768

(22) Filed: Feb. 4, 2020

(51) Int. Cl. G10L 15/26; G10L 15/20; G16H 10/60

(52) U.S. Cl. CPC G10L 15/265; G16H 10/60; G10L 15/20

(57) ABSTRACT

A voice recording and label making apparatus for converting users voice into text format, designed to be placed on documents comprising; medical documents, devices, X-rays, medical files, MRI etc. The first part of the apparatus comprise of a computing device that may receive input, the second part comprise of a device that may convert audio to formatted text and the third part comprise an apparatus that places the text format on a medical document or other documents of interest.

### BACKGROUND

### 1. Field

The present general inventive concept relates generally to a voice recording and label making system.

### 2. Description of the Related Art

During appointments, dentist usually require another individual to be present during examinations to record measurements and later translate them into labels that may be applied to medical documents. Currently, on the market, there are dictation devices that record audio; however, they lack the ability to translate the audio into labels.

Therefore, there is a need for a system that allows a user's voice to be recorded and then converted into a label.

### SUMMARY

The present general inventive concept provides a voice recording and label making system.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a . . .

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a system to provide record a voice of a medical professional and to convert the recording into a label to be applied to at least one medical document, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

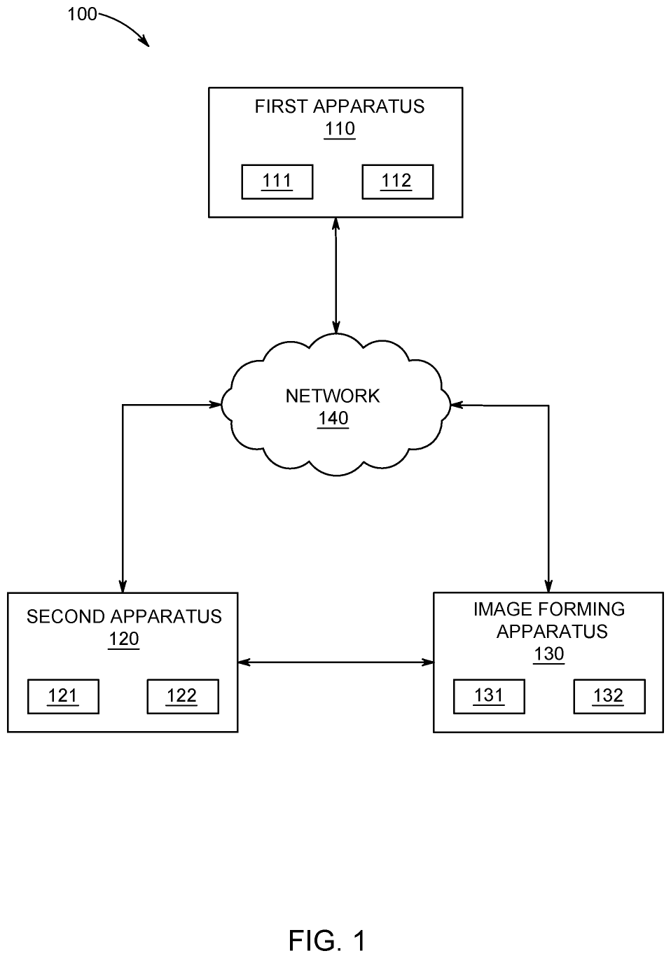

FIG. 1 illustrates a system 100 to provide record a voice of a medical professional and to convert the recording into a label to be applied to at least one medical document, according to an exemplary embodiment of the present general inventive concept.

The system 100 may include a first apparatus 110, a second apparatus 120, an image forming apparatus 130, and a network 140, but is not limited thereto, nor are all the components thereof described herein.

The first apparatus 110 may include a computing device that may receive input, transmit data, receive data, access the Internet, provide global positioning services (GPS), and communicate with other devices or systems via a network, and may include a desktop computer, a laptop computer, a tablet computer, and/or a mobile device, such as a cellular phone or a smart phone, but is not limited thereto.

The second apparatus 120 may include a computing device that may receive input, transmit data, receive data, access the Internet, provide global positioning services (GPS), and communicate with other devices or systems via a network, and may include a desktop computer, a laptop computer, a tablet computer, and/or a mobile device, such as a cellular phone or a smart phone, but is not limited thereto.

The first apparatus 110 and/or the second apparatus 120 may access the Internet to allow a user to access a website, and/or may allow a program and/or application to be executed thereon. The program and/or application may be designed to convert a voice of a user into a text format that may be printed onto a label that is specifically designed to be placed on a medical document, such as an x-ray, a medical file, an MRI, etc., but is not limited thereto.

For ease of description the first apparatus 110 will be hereinafter interpreted to be a mobile device, and the second apparatus 120 will be hereinafter interpreted to be a desktop computer.

The first apparatus 110 may include an input unit 111, such as a microphone, to allow the user to input audio data into the first apparatus 110 by speaking into or near the input unit 111, and a processor 112 to convert the input audio data into text data that is specifically formatted for the label of the medical document. The processor 112 may also include or may be connected to a storage unit to store the audio data and/or the converted text data, a central processing unit to process any type of data, and a communication unit to receive and/or transmit any type of data from and/or to the second apparatus 120 and/or the image forming apparatus 130 via the network 140. The first apparatus 110 may also include a display unit to display the data thereon, and a touch screen and/or keyboard/keypad to allow further input of data into the first apparatus 110.

The second apparatus 120 may include an input unit 121, such as a microphone, to allow the user to input audio data into the first apparatus 120 by speaking into or near the input unit 121, and a processor 122 to convert the input audio data into text data that is specifically formatted for the label of the medical document. The processor 122 may also include or may be connected to a storage unit to store the audio data and/or the converted text data, a central processing unit to process any type of data, and a communication unit to receive and/or transmit any type of data from and/or to the first apparatus 110 and/or the image forming apparatus 130 via the network 140. The second apparatus 120 may also include a display unit to display the data thereon, and a touch screen and/or keyboard/keypad to allow further input of data into the first apparatus 120.

As such, the user (e.g., a doctor) may begin speaking, and the first apparatus 110 and/or the second apparatus 120 may begin automatically recording a voice of the user. The processor 112 and/or the processor 122 may convert the input audio data into text data that is specifically formatted for the label of the medical document, and may send the text data to the image forming apparatus 130, which then prints out the label including the text data onto an x-ray or any other type of medical document desired by the user.

Alternatively, the user (e.g., a doctor) may begin speaking, and the first apparatus 110 may begin automatically recording a voice of the user. The processor 112 may convert the input audio data into text data that is specifically formatted for the label of the medical document, and may send the text data to the second apparatus 120, which may display the text data on a display screen thereof to allow the user to manipulate the text data onto an x-ray or medical document displayed on the display screen. Then, the user may send the displayed x-ray or medical document to the image forming apparatus 130, which then may print the x-ray or medical document.

Referring to FIG. 1, the network 140 may be at least one of the Internet, a cellular network, a universal mobile telecommunications systems (UMTS) network, a Long Term Evolution (LTE) network, a Global System for Mobile Communications (GSM) network, a local area network (LAN), a virtual private network (VPN) coupled to the LAN, a private cellular network, a private telephone network, a private computer network, a private packet switching network, a private line switching network, a private wide area network (WAN), a corporate network, or any number of private networks that can be referred to as an Intranet. The network 140 can be implemented with any number of hardware and software components, transmission media, and network protocols. FIG. 1 illustrates the network 140 as a single network, but is not limited thereto.

The first apparatus 110 and the second apparatus 120 can both can send data to, and receive data from each other over via the Internet or any of the above-mentioned networks. The first apparatus 110 and the second apparatus 120 can be directly coupled to the image forming apparatus 130. Alternatively, the first apparatus 110 and the second apparatus 120 can be connected to the image forming apparatus 130 via any other suitable device, communication network, or combination thereof. For example, the first apparatus 110 can be coupled to the image forming apparatus 130 via routers, switches, access points, and/or communication networks. In other words, the image forming apparatus 130, the apparatus 110, and the apparatus 120 may all communicate with each other via the network 140.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A voice recording and label making apparatus for translating words into labels on documents; the voice recording and label apparatus comprise of:
A voice of a medical professional or professional.
An apparatus that converts the professional's voice into labels.
An apparatus for placing labels on documents comprising of; medical documents, intellectual documents, X-ray and other medical documents or teaching documents.
